(19) United States
(12) Patent Application Publication
(10) Pub. No.: US 2020/0013484 A1
(43) Pub. Date: Jan. 9, 2020

(54) MACHINE LEARNING VARIANT SOURCE ASSIGNMENT

(71) Applicant: GRAIL, INC., Menlo Park, CA (US)

(72) Inventors: Archana Shenoy, Menlo Park, CA (US); Earl Hubbell, Palo Alto, CA (US)

(73) Assignee: GRAIL, INC., Menlo Park, CA (US)

(21) Appl. No.: 16/460,588

(22) Filed: Jul. 2, 2019

Related U.S. Application Data
(60) Provisional application No. 62/694,375, filed on Jul. 5, 2018.

(51) Int. Cl. G16B 40/20; G16B 30/10; G06N 5/04; G06N 20/00

(52) U.S. Cl. CPC G16B 40/20; G06N 20/00; G06N 5/04; G16B 30/10

(57) ABSTRACT

Systems and methods for determining a source of a variant include receiving a plurality of variants obtained from a biological sample, the variants being of unknown source upon receipt, and receiving, for each of the variants, a plurality of values for a plurality of covariates from the biological sample. The variants are input into a source assignment classifier to determine a source for each of the variants, the source being one of a plurality of possible sources. The source assignment classifier includes a plurality of coefficients associated with the plurality of covariates and a function that receives as input the values associated with each variant and the coefficients and outputs the determined source of each of the variants.

### CROSS-REFERENCE TO RELATED APPLICATIONS

This application claims the benefit of priority to U.S. Provisional Patent Application No. 62/694,375, filed on Jul. 5, 2018, and entitled “MACHINE LEARNING VARIANT SOURCE ASSIGNMENT,” the contents of which are herein incorporated by reference in their entirety.

### TECHNICAL FIELD

This disclosure generally relates to identification of cancer in a subject, and more specifically to performing a physical assay on a test sample obtained from the subject, as well as statistical analysis of the results of the physical assay.

### BACKGROUND

Analysis of circulating cell-free nucleotides, such as cell-free DNA (cfDNA) or cell-free RNA (cfRNA), using next generation sequencing (NGS) is recognized as a valuable tool for detection and diagnosis of cancer. Analyzing cfDNA can be advantageous in comparison to traditional tumor biopsy methods; however, identifying cancer-indicative signals in tumor-derived cfDNA faces distinct challenges, especially for purposes such as early detection of cancer where the cancer-indicative signals are not yet pronounced. As one example, it may be difficult to achieve the necessary sequencing depth of tumor-derived fragments. As another example, errors introduced during sample preparation and sequencing can make accurate identification cancer-indicative signals difficult. The combination of these various challenges stand in the way of accurately predicting, with sufficient sensitivity and specificity, characteristics of cancer in a subject through the use of cfDNA obtained from the subject.

### BRIEF SUMMARY

In some embodiments, a method includes receiving a plurality of variants obtained from a biological sample, the variants being of unknown source upon receipt; receiving, for each of the variants, a plurality of values for a plurality of covariates from the biological sample; and inputting the variants into a source assignment classifier to determine a source for each of the variants, the source being one of a plurality of possible sources. The source assignment classifier can include a plurality of coefficients associated with the plurality of covariates and a function receiving as input the values associated with each variant and the coefficients and outputting the determined source of each of the variants. The method can include providing the determined sources for the variants.

In some examples, determining the source for each of the variants comprises determining a numerical score for the determined source associated with each variant. In some examples, determining the source for each of the variants further comprises determining a numerical confidence value associated with the numerical score. In some examples, determining the source for each of the variants further comprises determining a numerical score for each of the possible sources. Further, in some examples, determining the source for each of the variants further comprises determining a numerical confidence value associated with each of the numerical scores associated with each of the possible sources.

In some examples, the plurality of sources include at least one of: a tumor source, a germline source, a blood source, an other source, and an unknown source. The source assignment classifier can assign variants to the unknown source where confidences in other sources are below at least one threshold.

In some examples, the values include information regarding cfDNA sequencing data, and the covariates includes at least one covariate indicating an accuracy of cfDNA sequencing for the variants. One of the covariates can be a count of variant reads in cfDNA. One of the covariates can be a count of reference reads in cfDNA. One of the covariates can be a total count of reference reads and variant reads in cfDNA. One of the covariates can be a variant allele frequency in cfDNA of one of the variants input into the model. One of the covariates can be a cfDNA sequencing quality score derived from a noise model. Further, one of the covariates can be a strand bias in the cfDNA sequencing quality score.

In some examples, the values include information regarding gDNA sequencing data, and the covariates includes at least one covariate indicating an accuracy of gDNA sequencing for the variants. One of the covariates can be a count of variant reads in gDNA. One of the covariates can be a count of reference reads in gDNA. One of the covariates can be a total count of reference reads and variant reads in gDNA. One of the covariates can be a variant allele frequency in gDNA. One of the covariates can be a gDNA sequencing quality score derived from a noise model. One of the covariates can indicate a presence or absence of the variant in gDNA greater than a threshold. One of the covariates can be a count of reads of a variant with overlapping positions in pileup data in gDNA. One of the covariates can be a count of variant reads in pileup data in gDNA. One of the covariates can be a count of reference reads in pileup data in gDNA. One of the covariates can indicate if one of the variants recurs above a threshold frequency. One of the covariates can be a ratio of a variant allele frequency in gDNA to a variant allele frequency in cfDNA, or vice versa. One of the covariates can indicate a category of an allele change from one allele to another allele. One of the covariates can indicate a trinucleotide context of one of the variants. One of the covariates can indicate a presence or absence of a variant in an external database of known cancer mutations. One of the covariates can indicate subject ancestry. One of the covariates can indicate if a position of one of the variants overlaps a segmental duplication. One of the covariates can indicate if a gene associated with one of the variants overlaps a known clonal hematopoiesis gene. One of the covariates can indicate if a threshold number of mapping locations overlap a position of one of the variant.

In some examples, the biological sample is obtained from one or more blood, plasma, serum, urine, cerebrospinal fluid, fecal matter, saliva, pleural fluid, pericardial fluid, cervical swab, saliva, or peritoneal fluid sample.

In some embodiments described herein, a non-transitory computer-readable medium stores one or more programs, the one or more programs including instructions which, when executed by an electronic device including a processor, cause the device to perform any of the methods described herein.

In some embodiments described herein, an electronic device comprises one or more processors, a memory, and one or more programs. The one or more programs are stored in the memory and configured to be executed by the one or more processors, and the one or more programs include instructions for performing any of the methods described herein.

### BRIEF DESCRIPTION OF THE DRAWINGS

FIG. 1A depicts a flow process of a method for performing a sequencing assay to generate sequence reads, in accordance with various embodiments.

FIG. 1B is an example process for analyzing sequence reads generated by the assay of FIG. 1, in accordance with various embodiments.

FIG. 2 is an example ad-hoc method for variant source assignment, in accordance with various embodiments.

FIG. 3 is an illustration of an example random forest machine learning model for variant source assignment, in accordance with various embodiments.

FIG. 4 is flowchart of a method for determining a source of a variant using a source assignment classifier, in accordance with various embodiments.

FIG. 5 shows a fragment length analysis of a training data sample MSK-EL-0150, broken out by variant source, according to the ad hoc method discussed in FIG. 2, and in accordance with various embodiments.

FIG. 6 shows the ratio of cfDNA to gDNA allele frequency in the training data, in accordance with various embodiments.

FIG. 7 shows the ratio of cfDNA to gDNA quality scores in the training data, in accordance with various embodiments.

FIG. 8 shows fractions of two example trinucleotide contexts in the training data, in accordance with various embodiments.

FIG. 9 shows the fraction of variants in the training data indicating whether those variants have or do not have segmental duplication associated with their corresponding position in the genome, in accordance with various embodiments.

FIG. 10 supports the conclusion that most of the variants in the example training data set are correctly assigned, in accordance with various embodiments.

FIG. 11 shows a t-SNE dimensionally reduced representation of the values of the covariates in the training data set, in accordance with various embodiments.

FIG. 12 shows biopsy matched variants found in both gDNA and cfDNA, in accordance with various embodiments.

FIG. 13 shows the cancer types of the biopsy matched variants found in both gDNA and cfDNA as shown in FIG. 12, in accordance with various embodiments.

FIG. 14. is a table showing results of a random forest source assignment classifier, according to one example embodiment, in accordance with various embodiments.

FIG. 15 is an example graph showing the contribution of the covariates to the classifier's variant source assignment, according to one embodiment, in accordance with various embodiments.

FIG. 16 shows fragment length analysis of variants classified by the example classifier as originating from various sources, in accordance with various embodiments.

FIG. 17 shows fragment length analysis of variants assigned as originating from tumor by the example classifier, but assigned as germline and other sources by the ad-hoc model, in accordance with various embodiments.

FIG. 18 illustrates plots of decisions made by two ad-hoc filter flags in the ad-hoc model which led to the classification of other by the ad-hoc model for those variants classified as originating from tumor by the example classifier, in accordance with various embodiments.

FIG. 19 shows fragment length analyses of variants classified as other by the ad-hoc model and which were classified as tumor by the example classifier, in accordance with various embodiments.

FIG. 20 illustrates plots of decisions made by five other ad-hoc filter flags in the ad-hoc model which led to the classification of other by the ad-hoc model for those variants classified as originating from tumor by the example classifier, in accordance with various embodiments.

FIG. 21 shows the results of an example alternative ad-hoc model with the depth high flag and with the same example source assignment classifier as discussed in this example, in accordance with various embodiments.

FIG. 22 shows the results of the removal of gDNA covariates for this example classifier, in accordance with various embodiments.

FIG. 23 depicts a block diagram of an example computer system for implementing various systems and methods of the present invention.

The figures and the following description relate to preferred embodiments by way of illustration only. It should be noted that from the following discussion, alternative embodiments of the structures and methods disclosed herein will be readily recognized as viable alternatives that may be employed without departing from the principles of what is claimed.

Reference will now be made in detail to several embodiments, examples of which are illustrated in the accompanying figures. It is noted that wherever practicable similar or like reference numbers may be used in the figures and may indicate similar or like functionality.

### DETAILED DESCRIPTION

### I. Overview and Definitions

Variants observed from cfDNA sequencing can originate from a number of sources. Accurate assignment of the categories is helpful for downstream analyses, such as for detecting the presence or absence or stage of cancer, including particular types of cancer, as well as for detecting the presence or absence of other biological effects such as clonal hematopoeisis (CH). Sequence analysis that makes use of cfDNA can also make use of gDNA as well. If the same sample processing assay is used to process related cfDNA and gDNA samples, technical artifacts endemic to the assay will be often shared in the generated cfDNA and gDNA data, and so it can be difficult to separate out which variants originate from a technical artifact caused by the assay and which are due to other sources such as CH and somatic variants.

To help address this problem, this description relates to a source assignment classifier (sometimes referred to as simply “the classifier” for convenience of description) that improves on existing methods for variant source assignment in cfDNA. The classifier is able to perform source assignment with respect to a variety of source categories, including identifying novel somatic mutations (for example, those arising as a result of cancer), mutations arising out of clonal hematopoiesis, germline mutations, and other mutations. Here, the category of other mutations includes variants caused by technical artifacts arising out of sample processing.

As new data is received, the classifier's coefficients/critical values can be easily retrained to improve accuracy of source assignment, thereby reducing the need for hand tuning of the classifier to achieve optimal source assignment results. In some embodiments, the classifier is a random forest classifier, however in practice any one of a number of classification techniques can be used to perform the source assignment.

The term “individual” refers to a human individual. The term “healthy individual” refers to an individual presumed to not have a cancer or disease. The term “subject” refers to an individual who is known to have, or potentially has, a cancer or disease.

The term “sequence reads” refers to nucleotide sequences read from a sample obtained from an individual. Sequence reads can be obtained through various methods known in the art.

The term “read segment” or “read” refers to any nucleotide sequences including sequence reads obtained from an individual and/or nucleotide sequences derived from the initial sequence read from a sample obtained from an individual.

The term “single nucleotide variant” or “SNV” refers to a substitution of one nucleotide to a different nucleotide at a position (e.g., site) of a nucleotide sequence, e.g., a sequence read from an individual. A substitution from a first nucleobase X to a second nucleobase Y may be denoted as “X>Y.” For example, a cytosine to thymine SNV may be denoted as “C>T.”

The term “indel” refers to any insertion or deletion of one or more base pairs having a length and a position (which may also be referred to as an anchor position) in a sequence read. An insertion corresponds to a positive length, while a deletion corresponds to a negative length.

The term “mutation” refers to one or more SNVs or indels.

The term “true” or “true positive” refers to a mutation that indicates real biology, for example, presence of a potential cancer, disease, or germline mutation in an individual. True positives are tumor-derived mutations and are not caused by mutations naturally occurring in healthy individuals (e.g., recurrent mutations) or other sources of artifacts such as process errors during assay preparation of nucleic acid samples.

The term “false positive” refers to a mutation incorrectly determined to be a true positive.

The term “cell free nucleic acid,” “cell free DNA,” or “cfDNA” refers to nucleic acid fragments that circulate in an individual's body (e.g., bloodstream) and originate from one or more healthy cells or from one or more cancer cells. Additionally cfDNA can come from other sources such as viruses, fetuses, etc.

The term “genomic nucleic acid,” “genomic DNA,” or “gDNA” refers to nucleic acid including chromosomal DNA that originates from one or more cells obtained from a subject. In various embodiments, gDNA can be extracted from a cell derived from a blood cell lineage, such as a white blood cell. For purposes of this description, gDNA does not refer to germline DNA. Thus, gDNA (genomic DNA) could include somatic variants that can, for example, originate from blood-forming lineages.

The term “somatic variant” generally refers to alterations in DNA that occur after conception and which are not present within germline DNA. The source of a somatic variant can be from a tumor, or from another underlying source such as clonal hematopoiesis. The description below provides methods of identifying the sources of variants, and in some instances blood-derived variants such as those arising from clonal hematopoiesis are explicitly separated out from somatic variants arising from other sources such as from tumors.

The term “germline” and “germline DNA” refers to DNA present in germ cells (egg and sperm cells that join to form an embryo). Germline variants are variants with which a subject is born (i.e., inherited variants), and which do not originate from cell division during one's life time (i.e., somatic mutations or mutations arising from cell-division in blood-forming lineages).

The term “circulating tumor DNA” or “ctDNA” refers to nucleic acid fragments that originate from tumor cells or other types of cancer cells, which can be released into an individual's bloodstream as a result of biological processes such as apoptosis or necrosis of dying cells or actively released by viable tumor cells.

The term “alternative allele” or “ALT” refers to an allele having one or more mutations relative to a reference allele, e.g., corresponding to a known gene.

The term “sequencing depth” or “depth” refers to a total number of read segments from a sample obtained from an individual.

The term “alternate depth” or “AD” refers to a number of read segments in a sample that support an ALT, e.g., include mutations of the ALT.

The term “reference depth” refers to a number of read segments in a sample that include a reference allele at a candidate variant location.

The term “variant” or “true variant” refers to a mutated nucleotide base at a position in the genome. Such a variant can lead to the development and/or progression of cancer in an individual.

The term “candidate variant,” “called variant,” or “putative variant” refers to one or more detected nucleotide variants of a nucleotide sequence, for example, at a position in the genome that is determined to be mutated. Generally, a nucleotide base is deemed a called variant based on the presence of an alternative allele on sequence reads obtained from a sample, where the sequence reads each cross over the position in the genome. The source of a candidate variant may initially be unknown or uncertain. During processing, candidate variants may be associated with an expected source such as gDNA (e.g., blood-derived) or cells impacted by cancer (e.g., tumor-derived). Additionally, candidate variants may be called as true positives.

### II. Sample Processing

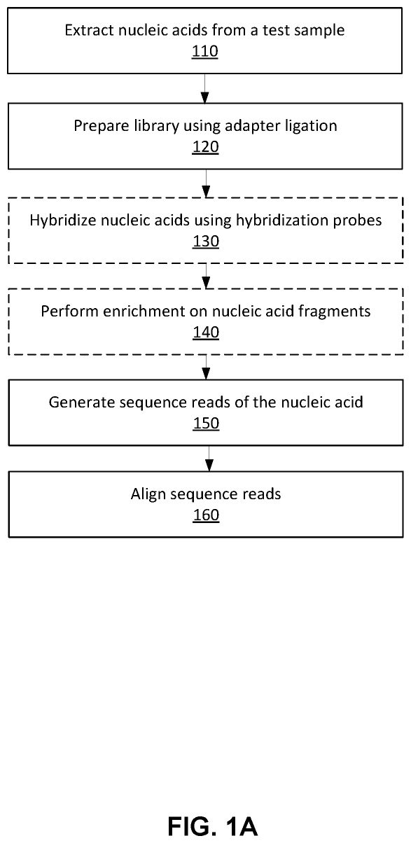

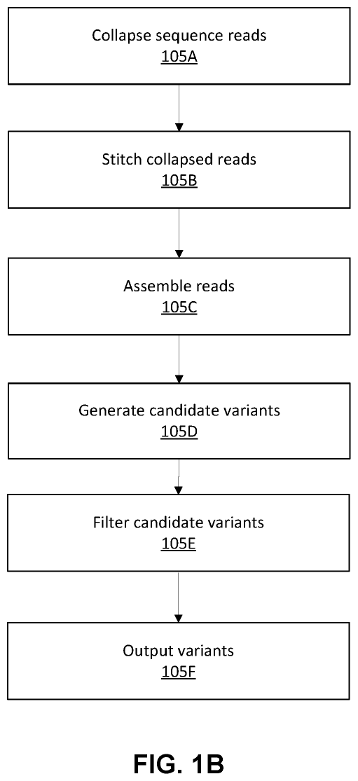

FIG. 1A is flowchart of a method for performing a physical assay (e.g., a sequencing assay) to generate sequence reads, in accordance with an embodiment. The method includes, but is not limited to, the following steps. For example, any step of the method can comprise a quantitation sub-step for quality control or other laboratory assay procedures known to one skilled in the art.

At step 110, nucleic acids (DNA or RNA) are extracted from a test sample. In the present disclosure, DNA and RNA can be used interchangeably unless otherwise indicated. That is, the following embodiments for using error source information in variant calling and quality control can be applicable to both DNA and RNA types of nucleic acid sequences. However, the examples described herein can focus on DNA for purposes of clarity and explanation. In various embodiments, DNA (e.g., cfDNA) is extracted from the test sample through a purification process. In general, any known method in the art can be used for purifying DNA. For example, nucleic acids can be isolated by pelleting and/or precipitating the nucleic acids in a tube. The extracted nucleic acids can include cfDNA or it can include gDNA, such as WBC DNA.

At step 120, a sequencing library is prepared. During library preparation, adapters, for example, include one or more sequencing oligonucleotides for use in subsequent cluster generation and/or sequencing (e.g., known P5 and P7 sequences for used in sequencing by synthesis (SBS) (Illumina, San Diego, Calif.)) are ligated to the ends of the nucleic acid fragments through adapter ligation. In some embodiments, unique molecular identifiers (UMI) are added to the extracted nucleic acids during adapter ligation. The UMIs are short nucleic acid sequences (e.g., 4-10 base pairs) that are added to ends of nucleic acids during adapter ligation. In some embodiments, UMIs are degenerate base pairs that serve as a unique tag that can be used to identify sequence reads obtained from nucleic acids. As described later, the UMIs can be further replicated along with the attached nucleic acids during amplification, which provides a way to identify sequence reads that originate from the same original nucleic acid segment in downstream analysis.

Optionally, a target gene panel can be used to high-depth sequence particular genomic regions. In such embodiments, optional steps 130 and 140 can be included in the sample processing pipeline. In step 130, hybridization probes are used to enrich a sequencing library for a selected set of nucleic acids. Hybridization probes can be designed to target and hybridize with targeted nucleic acid sequences to pull down and enrich targeted nucleic acid fragments that can be informative for the presence or absence of cancer (or disease), cancer status, or a cancer classification (e.g., cancer type or tissue of origin). In accordance with this step, a plurality of hybridization pull down probes can be used for a given target sequence or gene. The probes can range in length from about 40 to about 160 base pairs (bp), from about 60 to about 120 bp, or from about 70 bp to about 100 bp. In embodiments, the probes cover overlapping portions of the target region or gene. For targeted gene panel sequencing, the hybridization probes are designed to target and pull down nucleic acid fragments that derive from specific gene sequences that are included in the targeted gene panel. Alternatively, in the case of whole exome sequencing, hybridization probes are instead designed to target and pull down nucleic acid fragments that derive from exon sequences in a reference genome.

After a hybridization step 130, and again in the case of a targeted gene panel, the hybridized nucleic acid fragments are enriched 140. For example, the hybridized nucleic acid fragments can be captured and amplified using PCR. The target sequences can be enriched to obtain enriched sequences that can be subsequently sequenced. This improves the sequencing depth of sequence reads.

In step 150, the nucleic acids are sequenced to generate sequence reads. Sequence reads can be acquired by known means in the art. For example, a number of techniques and platforms obtain sequence reads directly from millions of individual nucleic acid (e.g., DNA such as cfDNA or gDNA) molecules in parallel. Such techniques can be suitable for performing any of targeted gene panel sequencing, whole exome sequencing, whole genome sequencing, targeted gene panel bisulfite sequencing, and whole genome bisulfite sequencing.

As a first example, sequencing-by-synthesis technologies rely on the detection of fluorescent nucleotides as they are incorporated into a nascent strand of DNA that is complementary to the template being sequenced. In some methods, oligonucleotides 30-50 bases in length are covalently anchored at the 5′ end to glass cover slips. These anchored strands perform two functions. First, they act as capture sites for the target template strands if the templates are configured with capture tails complementary to the surface-bound oligonucleotides. They also act as primers for the template directed primer extension that forms the basis of the sequence reading. The capture primers function as a fixed position site for sequence determination using multiple cycles of synthesis, detection, and chemical cleavage of the dye-linker to remove the dye. Each cycle consists of adding the polymerase/labeled nucleotide mixture, rinsing, imaging and cleavage of dye.

In an alternative method, polymerase is modified with a fluorescent donor molecule and immobilized on a glass slide, while each nucleotide is color-coded with an acceptor fluorescent moiety attached to a gamma-phosphate. The system detects the interaction between a fluorescently-tagged polymerase and a fluorescently modified nucleotide as the nucleotide becomes incorporated into the de novo chain.

Any suitable sequencing-by-synthesis platform can be used to identify mutations. Sequencing-by-synthesis platforms include the Genome Sequencers from Roche/454 Life Sciences, the GENOME ANALYZER from Illumina/SOLEXA, the SOLID system from Applied BioSystems, and the HELISCOPE system from Helicos Biosciences. Sequencing-by-synthesis platforms have also been described by Pacific BioSciences and VisiGen Biotechnologies. In some embodiments, a plurality of nucleic acid molecules being sequenced is bound to a support (e.g., solid support). To immobilize the nucleic acid on a support, a capture sequence/universal priming site can be added at the 3′ and/or 5′ end of the template. The nucleic acids can be bound to the support by hybridizing the capture sequence to a complementary sequence covalently attached to the support. The capture sequence (also referred to as a universal capture sequence) is a nucleic acid sequence complementary to a sequence attached to a support that can dually serve as a universal primer.

As an alternative to a capture sequence, a member of a coupling pair (such as, e.g., antibody/antigen, receptor/ligand, or the avidin-biotin pair) can be linked to each fragment to be captured on a surface coated with a respective second member of that coupling pair. Subsequent to the capture, the sequence can be analyzed, for example, by single molecule detection/sequencing, including template-dependent sequencing-by-synthesis. In sequencing-by-synthesis, the surface-bound molecule is exposed to a plurality of labeled nucleotide triphosphates in the presence of polymerase. The sequence of the template is determined by the order of labeled nucleotides incorporated into the 3′ end of the growing chain. This can be done in real time or can be done in a step-and-repeat mode. For real-time analysis, different optical labels to each nucleotide can be incorporated and multiple lasers can be utilized for stimulation of incorporated nucleotides.

Massively parallel sequencing or next generation sequencing (NGS) techniques include synthesis technology, pyrosequencing, ion semiconductor technology, single-molecule real-time sequencing, sequencing by ligation, nanopore sequencing, or paired-end sequencing. Examples of massively parallel sequencing platforms are the Illumina HISEQ or MISEQ, ION PERSONAL GENOME MACHINE, the PACBIO RSII sequencer or SEQUEL System, Qiagen's GENEREADER, and the Oxford MINION. Additional similar current massively parallel sequencing technologies can be used, as well as future generations of these technologies.

At step 160, the sequence reads can be aligned to a reference genome using known methods in the art to determine alignment position information. The alignment position information can indicate a beginning position and an end position of a region in the reference genome that corresponds to a beginning nucleotide base and end nucleotide base of a given sequence read. Alignment position information can also include sequence read length, which can be determined from the beginning position and end position. A region in the reference genome can be associated with a gene or a segment of a gene.

In various embodiments, a sequence read is comprised of a read pair denoted as R_1 and R_2. For example, the first read R_1 can be sequenced from a first end of a nucleic acid fragment whereas the second read R_2 can be sequenced from the second end of the nucleic acid fragment. Therefore, nucleotide base pairs of the first read R_1 and second read R_2 can be aligned consistently (e.g., in opposite orientations) with nucleotide bases of the reference genome. Alignment position information derived from the read pair R_1 and R_2 can include a beginning position in the reference genome that corresponds to an end of a first read (e.g., R_1) and an end position in the reference genome that corresponds to an end of a second read (e.g., R_2). In other words, the beginning position and end position in the reference genome represent the likely location within the reference genome to which the nucleic acid fragment corresponds. An output file having SAM (sequence alignment map) format or BAM (binary alignment map) format can be generated and output for further analysis.

Following step 160, the aligned sequence reads of the output file are passed to one or more computing devices for processing using a computational analysis, such as the source assignment classification method described in further detail below.

### III. Variant Identification

FIG. 1B is flowchart of a method for determining variants from sequence reads, in accordance with some embodiments. At step 105A, the processing system collapses aligned sequence reads. In some embodiments, collapsing sequence reads includes using UMIs, and optionally alignment position information from sequencing data of an output file to collapse multiple sequence reads into a consensus sequence for determining the most likely sequence of a nucleic acid fragment or a portion thereof. The unique sequence tag can be from about 4 to 20 nucleic acids in length. Since the UMIs are replicated with the ligated nucleic acid fragments through enrichment and PCR, the sequence processor can determine that certain sequence reads originated from the same molecule in a nucleic acid sample. In some embodiments, sequence reads that have the same or similar alignment position information (e.g., beginning and end positions within a threshold offset) and include a common UMI are collapsed, and the processing system generates a collapsed read (also referred to herein as a consensus read) to represent the nucleic acid fragment. The processing system designates a consensus read as “duplex” if the corresponding pair of collapsed reads have a common UMI, which indicates that both positive and negative strands of the originating nucleic acid molecule is captured; otherwise, the collapsed read is designated “non-duplex.” In some embodiments, the processing system can perform other types of error correction on sequence reads as an alternative to, or in addition to, collapsing sequence reads.

At step 105B, the processing system stitches the collapsed reads based on the corresponding alignment position information. In some embodiments, the processing system compares alignment position information between a first sequence read and a second sequence read to determine whether nucleotide base pairs of the first and second sequence reads overlap in the reference genome. In some use cases, responsive to determining that an overlap (e.g., of a given number of nucleotide bases) between the first and second sequence reads is greater than a threshold length (e.g., threshold number of nucleotide bases), the processing system designates the first and second sequence reads as “stitched”; otherwise, the collapsed reads are designated “unstitched.” In some embodiments, a first and second sequence read are stitched if the overlap is greater than the threshold length and if the overlap is not a sliding overlap. For example, a sliding overlap can include a homopolymer run (e.g., a single repeating nucleotide base), a dinucleotide run (e.g., two-nucleotide base sequence), or a trinucleotide run (e.g., three-nucleotide base sequence), where the homopolymer run, dinucleotide run, or trinucleotide run has at least a threshold length of base pairs.

At step 105C, the processing system assembles reads into paths. In some embodiments, the processing system assembles reads to generate a directed graph, for example, a de Bruijn graph, for a target region (e.g., a gene). Unidirectional edges of the directed graph represent sequences of k nucleotide bases (also referred to herein as “k-mers”) in the target region, and the edges are connected by vertices (or nodes). The processing system aligns collapsed reads to a directed graph such that any of the collapsed reads can be represented in order by a subset of the edges and corresponding vertices.

In some embodiments, the processing system determines sets of parameters describing directed graphs and processes directed graphs. Additionally, the set of parameters can include a count of successfully aligned k-mers from collapsed reads to a k-mer represented by a node or edge in the directed graph. The processing system stores directed graphs and corresponding sets of parameters, which can be retrieved to update graphs or generate new graphs. For instance, the processing system can generate a compressed version of a directed graph (e.g., or modify an existing graph) based on the set of parameters. In some use cases, in order to filter out data of a directed graph having lower levels of importance, the processing system removes (e.g., “trims” or “prunes”) nodes or edges having a count less than a threshold value, and maintains nodes or edges having counts greater than or equal to the threshold value.

At step 105D, the processing system generates candidate variants from the assembled paths. In some embodiments, the processing system generates the candidate variants by comparing a directed graph (which may have been compressed by pruning edges or nodes in step 105B) to a reference sequence of a target region of a genome. The processing system can align edges of the directed graph to the reference sequence, and records the genomic positions of mismatched edges and mismatched nucleotide bases adjacent to the edges as the locations of candidate variants. In some embodiments, the genomic positions of mismatched edges and mismatched nucleotide bases to the left and right of edges are recorded as the locations of called variants. Additionally, the processing system can generate candidate variants based on the sequencing depth of a target region. In particular, the processing system can be more confident in identifying variants in target regions that have greater sequencing depth, for example, because a greater number of sequence reads help to resolve (e.g., using redundancies) mismatches or other base pair variations between sequences.

At step 105E, the processing system filters the candidate variants using one or more types of models or filters. For example, the processing system scores the candidate variants using a noise model, a joint model, an edge variant prediction model, or corresponding likelihoods of true positives or quality scores. More detail about the filtering of these candidate variants is described in U.S. Provisional Application Ser. No. 62/642,301, incorporated by reference further below in this description.

Returning to FIG. 1B, at step 105F, the processing system outputs the filtered candidate variants.

### IV. Ad-Hoc Source Assignment Method

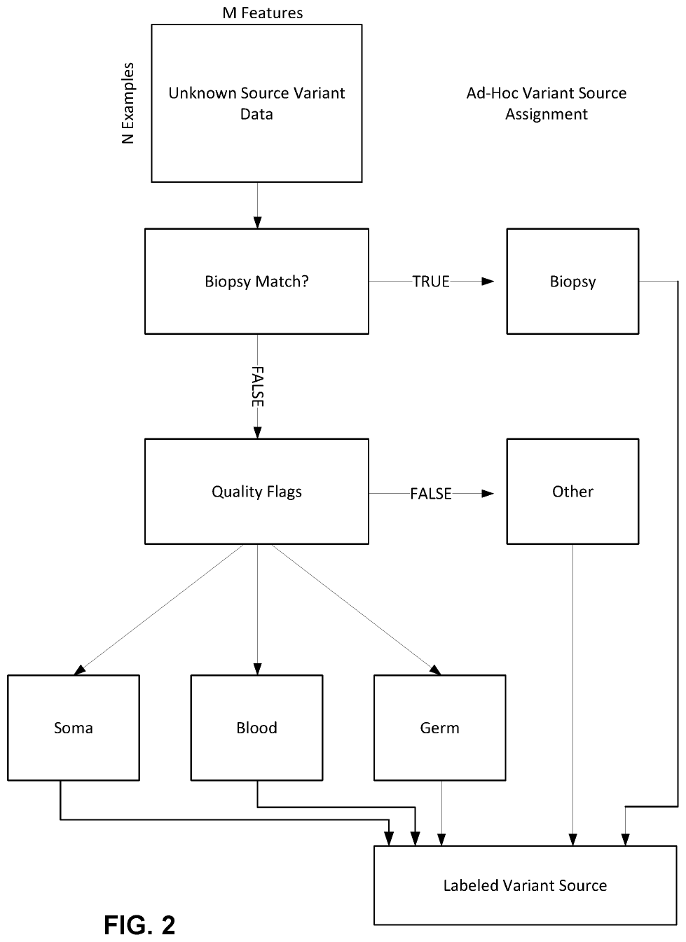

FIG. 2 is an example ad-hoc method for variant source assignment, according to some embodiments. This example ad-hoc method is a heuristic method designed to assign sources based on biological considerations which can be easily evaluated given a set of cfDNA and gDNA sequencing data obtained from a sample. In some examples, this method requires hand-tuning of the particular parameter values used, which can be complicated as more data sets are processed and as underlying factors, such as the method of sample processing, change over time. In some cases, its simplicity also means that it has naturally limited sensitivity, in which case it can be unable to correctly assign all variants unless more sophisticated (also hand-tuned) heuristics are added.

The ad-hoc method is useful as a tool for obtaining initial labeled training data that can be used to train a machine learned classifier such as the source assignment classifier. In such embodiments, the ad hoc method can assume access to separate biopsy samples that correspond to the cfDNA and gDNA samples obtained from a subject and which indicate exactly which mutations are novel somatic mutations such as may arise from cancer. Together, sequencing data from biopsy samples, cfDNA sequence data, and gDNA sequence data can be used to label the training data for the source assignment classifier.

In some embodiments, the ad-hoc source assignment method assigns sources as follows: The source of a variant is labeled as originating from cancer (cancer source) if the variant is matched in sequencing data from a biopsy sample obtained from a subject.

The source of a variant is labeled as “other” if it is flagged as failing any one or more of the following quality criteria (though this could be done in inverse, such that it is labeled as other if it meets the inverse of any of these criteria). Example quality criteria: depth high (>90^{th} quantile for a given subject as compared to other genomic positions for that subject), depth low (<10^{th} quantile/subject), cfDNA strict (AF >0.001, reads containing the variant allele >=3); multiple alleles flag which returns true if multiple different alleles are called at the same position in reads in a given subject, outlier bed flag. The other category includes technical artifacts, such as those that arising out of sample processing. Other predictors such as strand bias or location of variant alleles within reads could also be incorporated into this ad-hoc procedure for assignment into the other category.

In the following three example source assignment decisions, AND is interpreted as a logical AND.

The source of a variant is labeled as “germline” if the following logical operation is met (returns true):
gDNA of >0.15 & gDNA_strict (at least 3 alternate allele reads of 0.1% frequency or above) AND
cfDNA of >0.35 AND
the variant at that position is flagged (true or false) as a recurrent variant in many samples in the populations

The source of a variant is labeled as “blood” (blood derived variant sources such as clonal hematopoiesis) if the following logical operation is met (returns true):
ratio of cfDNA/gdna reads <3 AND
present in both cfDNA and gDNA (greater than some de minimis threshold) AND
source not already labeled as germline

The source of a variant is labeled as “somatic” if the following logical operation is met (returns true):
variant not already labeled as source “blood” AND
no match for the variant in gDNA (less than some de minimis threshold) AND
source not already labeled as germline

### V. Source Assignment Classifier Training

In contrast to the ad-hoc source assignment method, the source assignment classifier uses a supervised machine learning model to assign sources to variants. The classifier includes a set of covariates (also referred to as parameters or features), and a corresponding set of trained coefficients (also referred to as critical values) for those covariates. The classifier further includes a function which receives as inputs values for the covariates from a set of data associated with a blood sample of a subject, and together with the function and the critical values, generates an output prediction for each variant. This output prediction can take the form of a single source assignment, or it can take the form of a set of scores representing numerical likelihoods (e.g. a vector of values) that variant is associated with each of a set of possible sources. Each of these scores can also be associated with a numerical confidence value indicating the classifier's confidence in its determination of the associated score.

The covariates used in the model can vary by implementation. Examples of covariates that can be used as a part of the source assignment classifier are listed in Table 1 below. Additional covariates that are variants of these covariates or which are not listed below can also be used in the source assignment classifier. The left-hand column represents a shorthand name for the covariate, and the right-hand column explains the covariate and provides (in brackets) an example of the numerical units or categorical labels the covariates value can take on from received data.

TABLE 1
Covariates
covariates from cfDNA sequencing

| | |
|---|---|
| cfdna_variant_reads | number of variant reads in cfdna [count] |
| cfdna_ref_reads | number of reference reads in cfdna [count] |
| cfdna_depth | cfdna_variant_reads + cfdna_ref_reads [count] |
| cfdna_af | variant allele frequency in cfdna data [0-1] |
| cfdna_qual_score | cfdna quality score (>60) derived from noise modeling |
| | [unitless number]; the calculation of the quality score can be |
| | found in U.S. Provisional Application Ser. No. 62/642,301, |
| | incorporated by reference below; as an example: the quality |
| | score can be a Phred quality score Q = −10 · log₁₀ P, where P is |
| | the likelihood of an incorrect candidate variant call (e.g., a |
| | false positive) |

covariates from gDNA sequencing

| | |
|---|---|
| gdna_variant_reads | number of variant reads in gdna [count] |
| gdna_ref_reads | number of reference reads in gdna [count] |
| gdna_depth | gdna_variant_reads + gdna_ref_reads [count] |
| gdna_af | variant allele frequency in gdna data [0-1] |
| gdna_var_present | presence/absence of variant reads in WBC, true if greater than |
| | a threshold number of reads with variant in gDNA [binary] |
| gdna_qual_score | gdna quality score derived from noise modeling [unitless |
| | number] |
| pileup_gdna_depth | In some embodiments, a pileup file contains for each position |
| | the number of reads for each possible variant. In some |
| | implementations of the variant caller, if a particular variant |
| | seen in cfDNA was not called in gDNA by the variant caller, |
| | then the gDNA depth was also not included in the “number of |
| | gdna reads” covariate. Thus, the pileup file is a source of the |
| | corresponding gDNA depth (pileup_gdna_depth) to |
| | differentiate between not getting any coverage in gDNA at that |
| | location vs getting coverage but not seeing a variant. [count] |
| pileup_gdna_variant_reads | number of variant reads in pileup data [count] |
| pileup_gdna_reference_reads | number of variant reads in pileup data [count] |

covariates from local learning of data

| | |
|---|---|
| flag_cfdna_recur | indicates if the variant at that position is recurrent in many |
| | samples in the population. When the variant occurs in a |
| | particular person, the flag is set to true or false depending on |
| | the recurrence rate. [binary] |
| cfdna_gdna_af_ratio | ratio of cfdna and gdna allele frequency [unitless number] |

covariates from biological annotations

| | |
|---|---|
| allele_change | represents which allele changed to which other allele. So, AT |
| | indicates a change from A to T, etc. this may be encoded a |
| | categorical variable, each possible change or indel being a |
| | different category. [AT, AG, AC, GA, GT, GC, TA, TG, TC, |
| | CA, CG, CT] |
| trinucleotide_context | 3 letter nucleotide sequence centered on variant position |
| | [categorical, one category for each trinucleotide context. |
| | Alternatively, could have a separate covariate for each |
| | trinucleotide context with a binary value] |
| in_cosmic | presence/absence in the Catalog of Somatic Mutations in |
| | Cancer (COSMIC). |
| | cancer<dot>sanger<dot>ac<dot>uk</>cosmic [binary] |
| exac_pop_af | population frequency in Exome Aggregation Consortium |
| | (ExAC) data for matched ancestry. |
| | exac<dot>broadinstitute<dot>org</> [0-1] |
| strand_bias_score | p-value for strand bias in cfdna sequences [0-1] |
| is_segdup | indicates if the position overlaps a segmental duplication |
| | [binary] |
| is_clonal_hemat | indicates if the gene that the variant is in overlaps a known |
| | clonal hematopoiesis gene (source |
| | www<dot>nejm<dot>org</>doi</>full</>10<dot>1056</>NEJ |
| | Moa1409405) [binary] |
| crg100, crg50, crg36 scores | minimum crg score overlapping the variant, where crg score is |
| | calculated as 1/# of mapping locations) [unitless number] |

Prior to use of the source assignment classifier, the critical values of the classifier are trained using a supervised learning technique. In some embodiments, this technique is a random forest classifier, however in other embodiments any one of a number of supervised learning techniques may be used instead. Examples include but are not limited to: gradient boosting machines, support vector machines, multinomial regressions, neural networks, etc.

In some embodiments, the source assignment classifier is trained using a set of training data where the values of the covariates are obtained from a training data set, and where the labels for the training data set were obtained using the ad hoc source assignment method discussed above. Performance of the source assignment classifier was determined using a held out set of samples from the training data set. In other embodiments, alternate source assignment methods can be used to assign source assignment training labels to the training data. For example, the joint caller of U.S. Provisional Application Ser. No. 62/642,301 can be used to assign sources to training data, the contents of which are incorporated by reference herein in their entirety. As another example, the selection coefficient of U.S. Provisional Application No. 62/673,779 can be used to assign sources to training data, the contents of which are incorporated by reference herein in their entirety.

### V.A. Random Forest Classifier Example

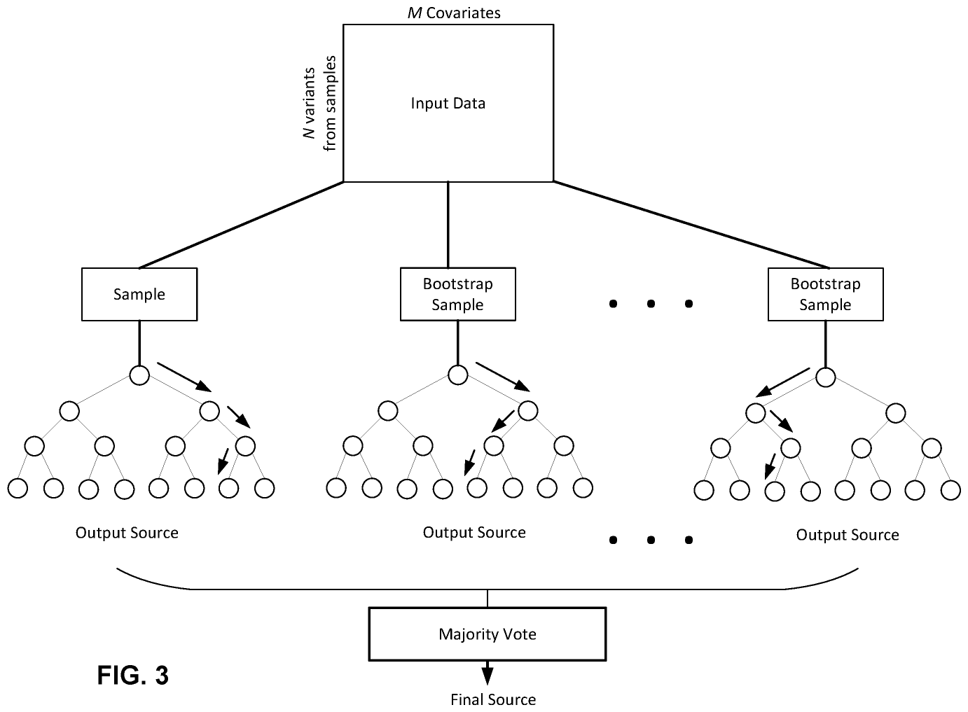

FIG. 3 is an illustration of an example random forest machine learning model for variant source assignment, according to some embodiments. A random forest classifier (RFC) is an ensemble method involving a collection of distinct decision trees, each of which classifies a source of a variant by testing the entry through a sequence of covariate conditions. The decisions of the individual trees are combined to determine a final decision as to the source of the variant. Decision trees provide an easy and intuitive way of interpreting the classification of data items.

The structure of each decision tree can depend on a number of factors, examples of which include but are not limited to the number of decision trees in total, size (depth) of the tree representing the number of covariates considered in each tree, the minimum size of terminal nodes of the trees, and the maximum number of terminal nodes.

Each decision tree includes a hierarchical structure with a plurality of nodes and a plurality of directed edges between a parent node and each of a plurality of child nodes. The split at each parent node between the child nodes is based on a test of one of the covariates that compares a value of the covariate in the training data against a critical value to determine whether the value meets the critical value or not.

The training process iterates through possible critical values for the covariates of the decision trees to determine critical values that meaningfully separate the training data based on the known labels of the training data. Ideally, the critical values will be determined such that the final determination of the source assignment classifier is able to appropriately determine the source of variants from the test data, held out validation test data, and data for which the source of a variant is not known.

During training, the critical value is iterated on over many instances of training data (i.e., individual variants in the training data) to maximize an improvement function at each split within each of the decision trees. Generally, the optimal condition for each split is where one partition predominantly has entries with one classification label (e.g., “0”), and the other partition predominantly has entries with the other classification label (e.g., “1”). During training, various partitions of the training data are generated based on a multitude of possible values for the various covariates. Further, each leaf node of a decision tree is assigned a source classification label corresponding to the label of the training data and based on the contents of the data partition at that leaf node. An improvement metric is calculated from the improvement function, and critical value are selected for the covariates based on the highest improvement metric identified for each split.

The improvement function is based on an impurity function that measures the “purity” of a test partition, or how predominant one classification label is among the entries in the partition. The impurity function retains a low value when a partition includes a high proportion of entries from one classification label, and a high value when the partition contains an almost equal combination of entries with both classification labels “0” and “1.” In some embodiments, the impurity function is the Gini impurity function, though in practice other impurity functions can be used.

In some embodiments, the improvement function Δi(s,t) for a test covariate at node t for a split s can be given by:

Δi(s,t)=i(t)−π(l)·i(l)·π(r)·i(r)

where i(t) is the impurity function for node t, i(l) is the impurity function for the potential left child node of node t, i(r) is the impurity function for the potential right child node of node t, π(l) is the proportion of data entries sent to the left node from node t, and π(r) is the proportion of data entries sent to the right node from node t. The improvement function above measures the decrease in impurity if a subset of the training data at node t were to be split at s into child nodes l and r. The improvement function Δi(s,t) is maximized when the impurity function of the potential left and right child nodes are minimized. In other embodiments, the improvement function can be given by other impurity function-based functions, such as information gain, likelihood ratio, gain ratio, distance measure, and the DKM criterion, etc.

In some embodiments, during training decision trees grow in size, that is they add child nodes with additional covariate splits from parent nodes until the maximum of the improvement function for a split is less than a threshold. In the same or a different embodiment, large decision trees with many layers can use a separate validation data set to reduce or prune the size of the tree.

### V.B. Source Assignment Classifier Use

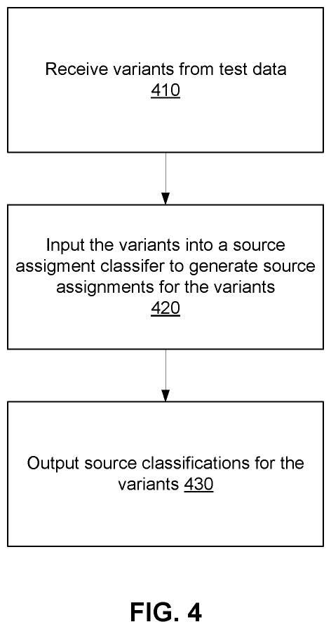

FIG. 4 is flowchart of a method for determining a source of a variant using a source assignment classifier, according to some embodiments. In use, for a received 410 variant in the test data to be evaluated, values for the covariates used in the decision trees of the trained source assignment classifier are input 420 into the source assignment classifier. The source assignment classifier 430 then outputs a source assignment for the variant.

The exact form of the output can vary by embodiment. In some embodiments, only the source assignment is output by the classifier. In some embodiments, the assignment is provided along with a numerical confidence value. In still some embodiments, a separate numerical score is output for each possible source, indicating the model's relative belief regarding the likelihood that each such source is the correct source. These separate scores can be accompanied by individual confidence values for each source and/or an overall confidence for the entire set of outputs.

In further embodiments, the source assignment classifier is a forest classifier including a number of trees. Each tree of the source assignment classifier outputs a numerical score indicating a likelihood that a variant originated from a particular source, along with a confidence value in the generated score. The scores and confidence values of the trees can be aggregated in some form, for example using a voting method or an aggregation function, to determine scores and confidence values for each source category.

In some embodiments, an additional source assignment category of “unknown” may be assigned if the confidence values for the other source categories are below one or more thresholds. A single threshold can be used such that if the scores for all other categories are below a single threshold, the unknown category is assigned to the variant. Alternatively, different thresholds can be determined for each category, or even for each tree.

This process is generally repeated for the many identified variants of the sample for each subject.

### VI. Examples

### VI.A. Example 0: Biopsy Sample Handling

The biopsy samples described in the example herein were run by Memorial Sloan Kettering Cancer Center (MSKCC) using their MSK-IMPACT protocol, including a targeted panel and somatic variant calling pipeline customized for tumor-normal pairs. More detail regarding this process can be found in “Mutational landscape of metastatic cancer revealed from prospective clinical sequencing of 10,000 Subjects;” Nature Medicine 23, 703-713 (2017) (www<dot>nature<dot>com</>articles</>nm<dot>4333)

Any other tumor sequencing and variant calling protocol of high quality may be used instead.

### VI.B. Example 1: Analysis of Ad-Hoc Source Assignment Method

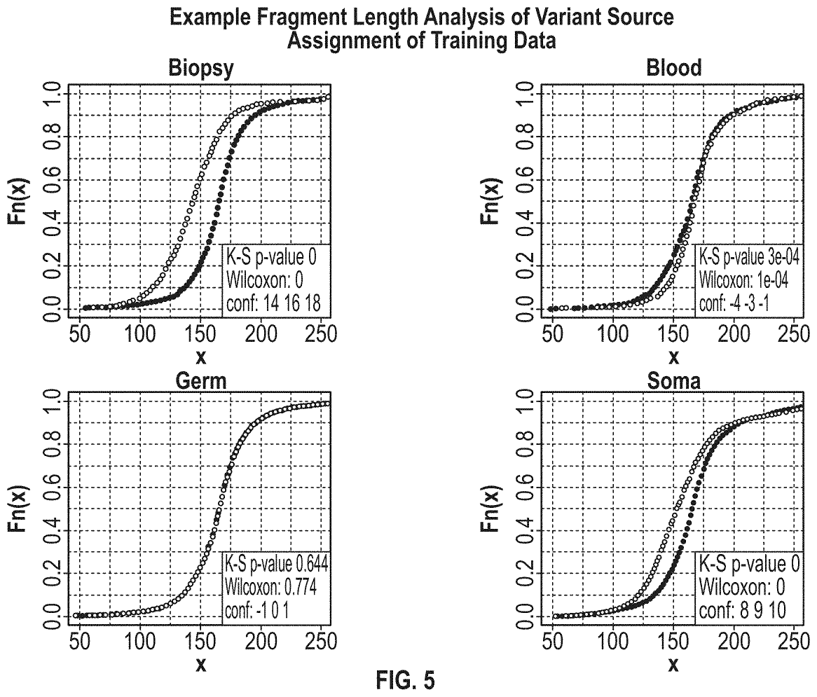

FIG. 5 shows a fragment length analysis of a training data sample MSK-EL-0150, broken out by variant source, according to the ad hoc method discussed in FIG. 2. Fragment length distributions are a useful measure of source classification as different sources of variants are known to exhibit different effects on fragment length distributions in cfDNA, particularly as compared to the fragment length distributions obtained directly from healthy or cancerous tissue. For example, tumor-shed cfDNA fragments are generally on the order of 10-20 base pairs shorter than healthy fragments obtained from normal tissue.

FIG. 5 contains four plots, one for each possible source (the source “other” has been omitted from this example). In all of these plots, the hollow (white) dots show the cumulative distribution of fragment lengths for fragments with the alternate allele/variant while the solid black points show the cumulative distribution of fragment lengths for fragments with the reference allele.

In the training data, the biopsy matched variants act as ground truth, as they are obtained directly from the cancerous tumor of the subject (in this example subject MSK-EL-0150).

In this example, the alternate variants measured in cfDNA (hollow dots, left hand curve of “Biopsy” graph) show an expected shift in cumulative fragment length distribution towards shorter fragment relative to the cumulative fragment length distribution for the reference allele (solid dots, right hand curve of “Biopsy” graph). Similarly, in this example novel somatic mutations show a similar expected shift in cumulative fragment length distribution towards shorter fractions (see, e.g., “Soma” graph of FIG. 5). This is expected as in a cancerous subject it is expected that the fragments appearing in cfDNA will be shorter on average.

Additionally, in this example blood match variants measured in cfDNA show a shift in cumulative fragment length distribution that is either neutral or slightly longer than those obtained for the reference allele, which is expected because variants caused by clonal hematopoiesis will be present in both the cfDNA as well as in gDNA (see, e.g., “Blood” graph of FIG. 5). Further, in this example germline matched variants show no fragment length shift, as expected, because the germline variants present in a subject will be present both in a tumor biopsy as well as in the cfDNA of healthy tissue (see, e.g., “Germ” graph of FIG. 5).

The example of FIG. 5 illustrates that the ad hoc source assignment method performs reasonably well with respect to this fragment-length distribution test. However, as discussed above the ad-hoc source assignment method is not scalable in that it requires hand-tuning of parameters for each new data set that is considered.

### VI.C. Example 2A: Analysis of Training Data Set and Application to Example Source Assignment Classifier

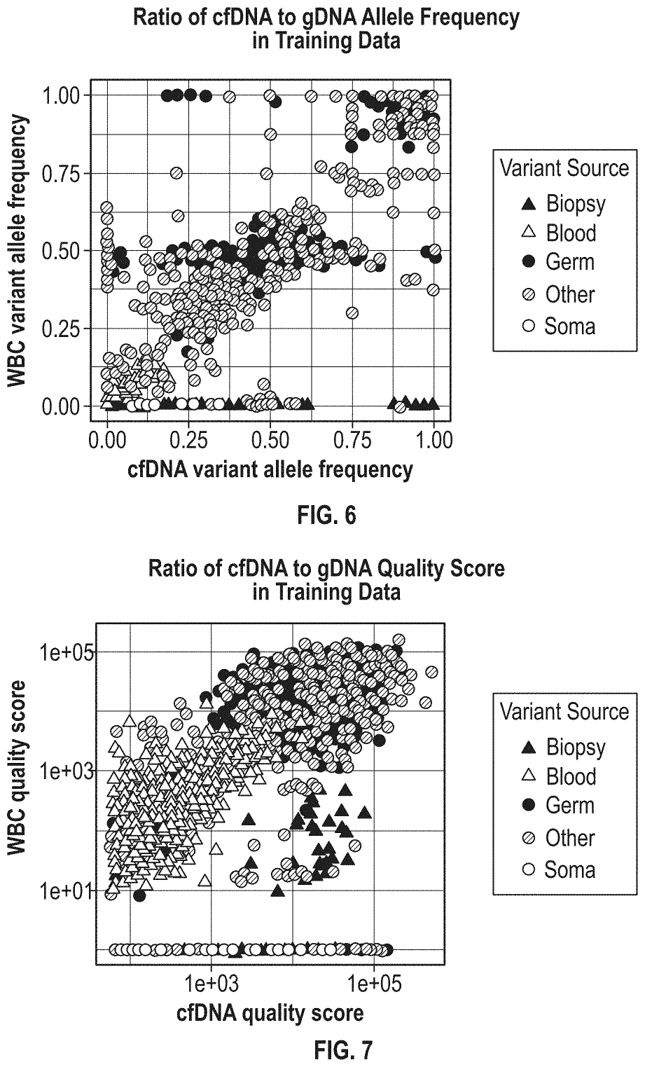

FIG. 6 shows the ratio of cfDNA to gDNA allele frequency in the training data. FIG. 7 shows the ratio of cfDNA to gDNA quality scores in the training data. During training of the example source assignment classifier, samples labeled as source germline or other by the ad-hoc model were downsampled. This downsampling helps avoid biasing the training of the source assignment classifier towards being overly proficient at recognizing germline and other mutations, which can occur at the expense of proficiency in recognizing novel somatic and blood originating variants.

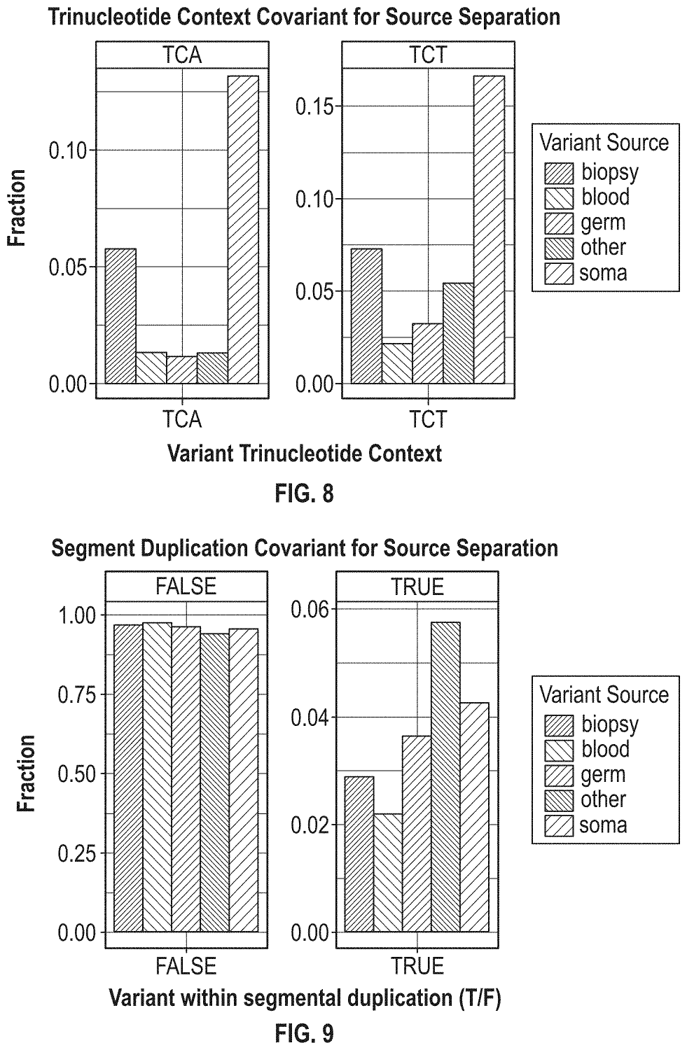

FIG. 8 shows fractions of two example trinucleotide contexts in the training data. FIG. 9 shows the fraction of variants in the training data indicating whether those variants have (true) or do not have (false) segmental duplication associated with their corresponding position in the genome. The plots of FIGS. 8 and 9 are indicative that the corresponding covariates (trinucleotide context and segmental duplication) are useful in the source assignment classifier for correctly classifying the sources of variants.

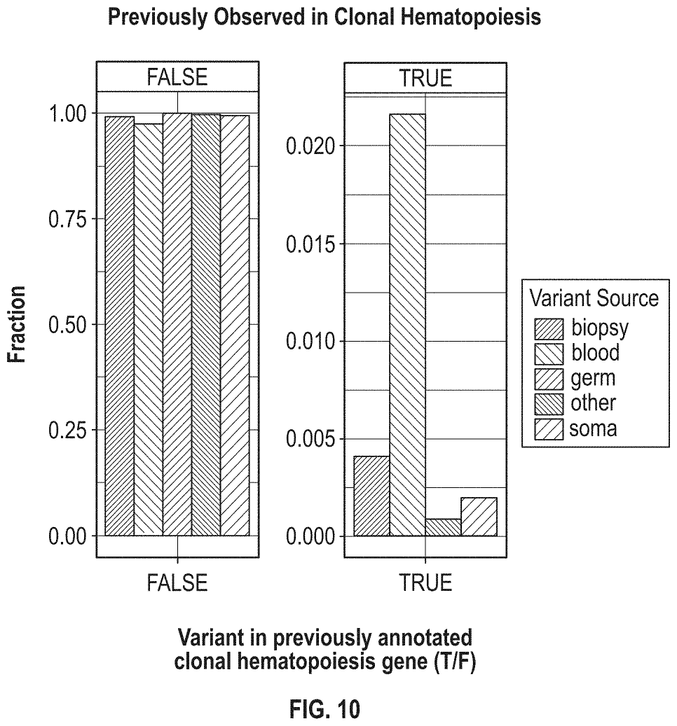

FIG. 10 supports the conclusion that most of the variants in the example training data set are correctly assigned. Specifically, it illustrates in the right hand plot (“TRUE” plot) that a measurable fraction of variants that are assigned as being blood derived using the ad hoc model (relative to variants assigned to other sources) come from previously identified clonal hematopoiesis genes. Additionally, the left hand plot (“FALSE” plot) has a higher fraction of total variants versus the right hand plot (y axis scale on left hand plot is higher than right hand plot). The heights of the various plots indicate a normalized count of the number of variants determined to be arising out of each source by the source assignment classifier.

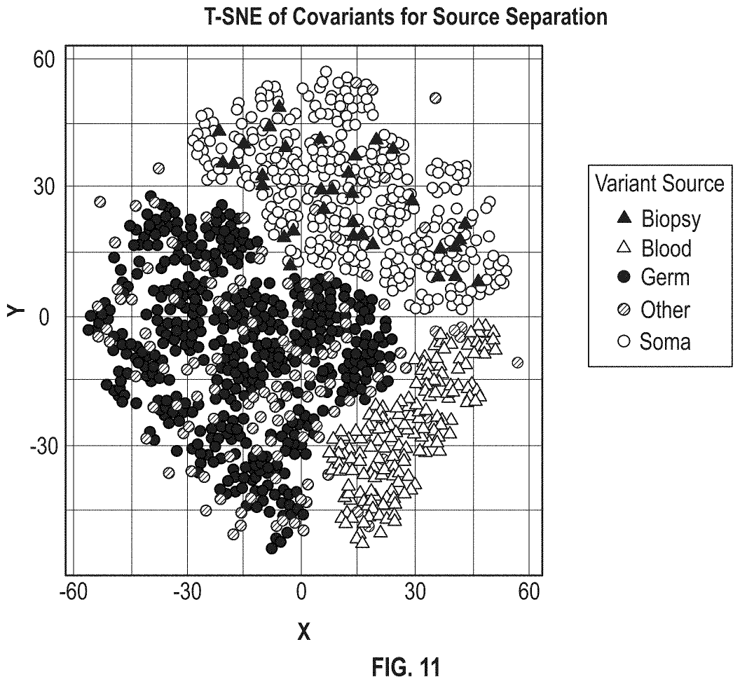

FIG. 11 shows a t-SNE dimensionally reduced representation of the values of the covariates in the training data set. Labels by source have been assigned by source assignment classifier separately according to the example 2, in this next subsection. The relatively clean separation by groups (except for the source “other” which generally refers to erroneous or unclassifiable data) therefore illustrates that the covariate data includes some latent information which the source assignment classifier is able to extract in order to classify variants by source.

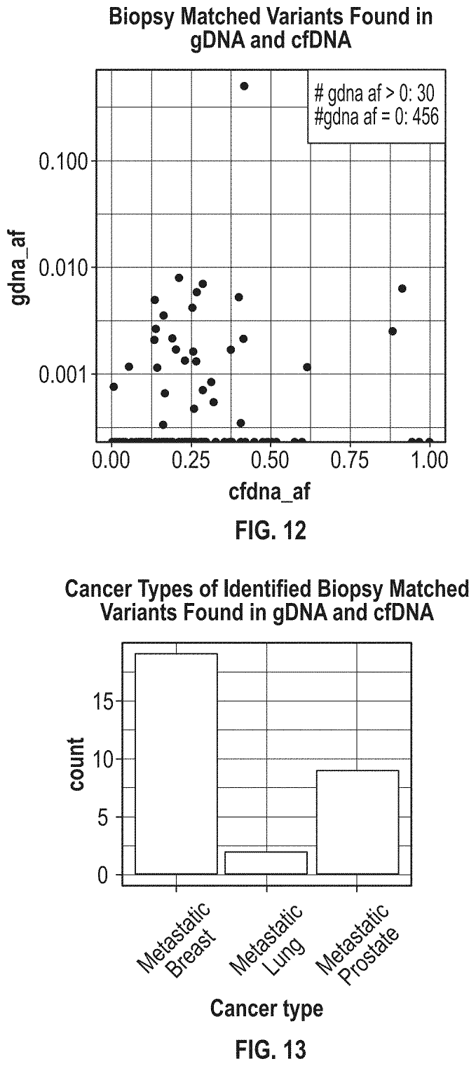

FIG. 12 shows biopsy matched variants found in both gDNA and cfDNA, according to one embodiment. FIG. 12 illustrates that although many biopsy matched variants had gDNA AF of zero, not all did. Consequently, a simple filter to assign a source of a variant as not from cancer simply because the variant was found in gDNA would be incorrect. Instead, although the gDNA read count or AF can be informative of the source of a variant, it is not a decisive factor on its own.

FIG. 13 shows the cancer types of the biopsy matched variants found in both gDNA and cfDNA as shown in FIG. 12. Thus, FIG. 13 illustrates that these variants occur in multiple different types of cancer, further illustrating that assigning a source of variant based on presence/absence of the variant in gDNA would be erroneous.

### VI.D. Example 2B: Example Source Assignment Classifier Results

In this example embodiment, the classifier was trained on single nucleotide variants from 18 chromosomes of samples using all of the covariates from Table 1. The training data set was separated such that 80% of the training data set was used for training the classifier, and the remaining 20% was held out for validation and is herein referred to as the test data set. Here, the sources for assignment were partially collapsed; the sources “biopsy” (biopsy matched variants) and “somatic” were combined into a single source referred to as “tumor.”

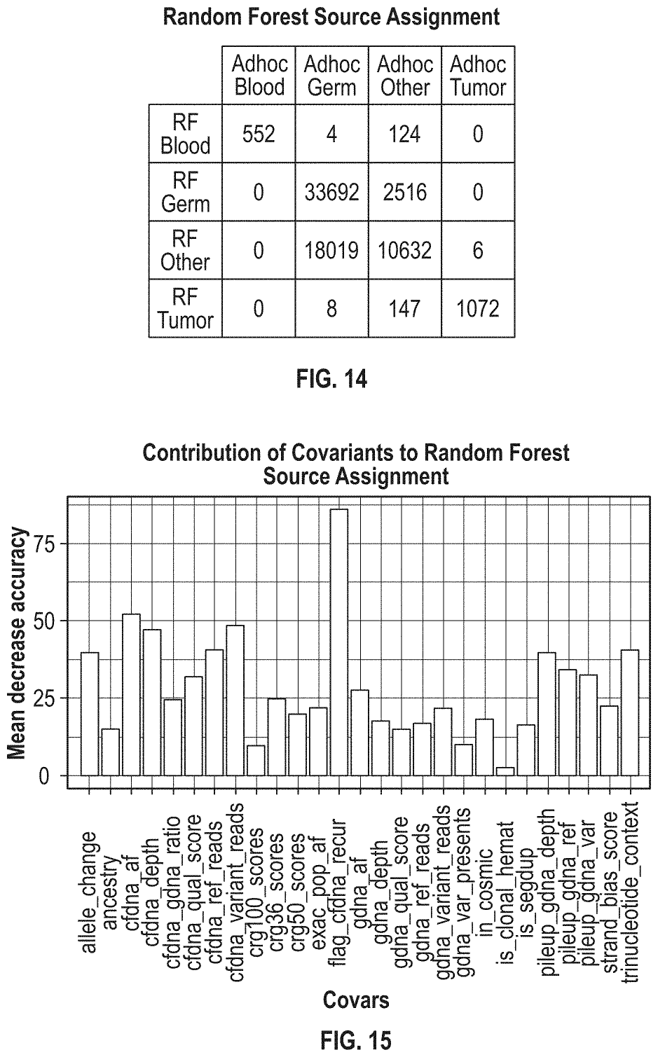

FIG. 14. is a table showing results of a random forest source assignment classifier, according to one example embodiment. In the table of FIG. 14, the columns represent variant source assignments predicted by the ad hoc model, and rows represent source assignment calls by the classifier. The vast majority of the data runs along the axis, suggesting at first cut that both models perform similarly within a certain level of performance. Further, these results further indicate that blood and tumor variants are being assigned with high accuracy by the classifier. Of interest is where the two models differ. As an example, those variants sourced as originating from tumor by the classifier but not by the ad hoc model (147) indicates circumstances where the classifier can be outperforming the ad hoc model.

FIG. 15 is an example graph showing the contribution of the covariates to the classifier's variant source assignment, according to one embodiment. The x-axis of this plot illustrates various covariates, and the y-axis indicates the level of impact each covariate had on the improvement of the classifier's performance as measured according to mean decrease in accuracy when that covariate is removed. Here, flag_cfdna_recur indicates that the recurrence of a variant in the population of samples is very helpful in properly determining the source of a variant.

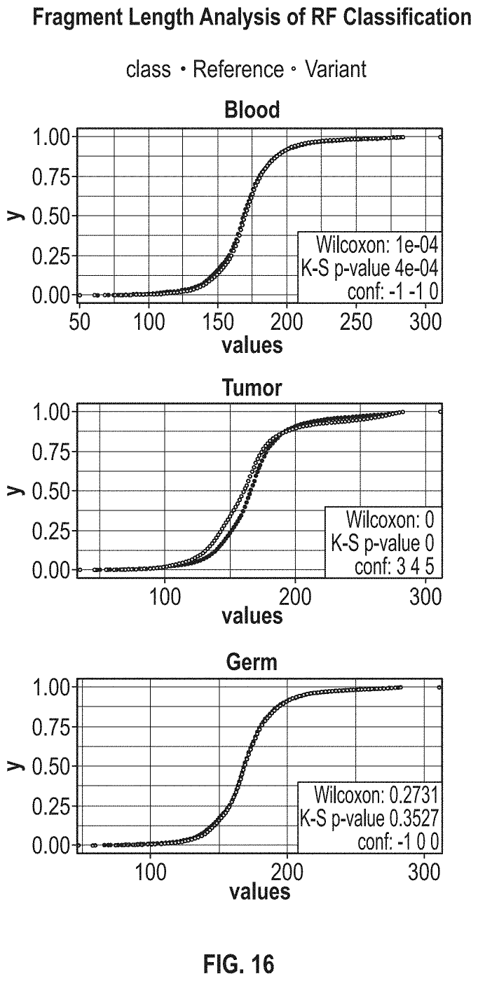

FIG. 16 shows fragment length analysis of variants classified by the example classifier as originating from various sources. FIG. 16 illustrates that the source assignment classifier classified sources as expected.

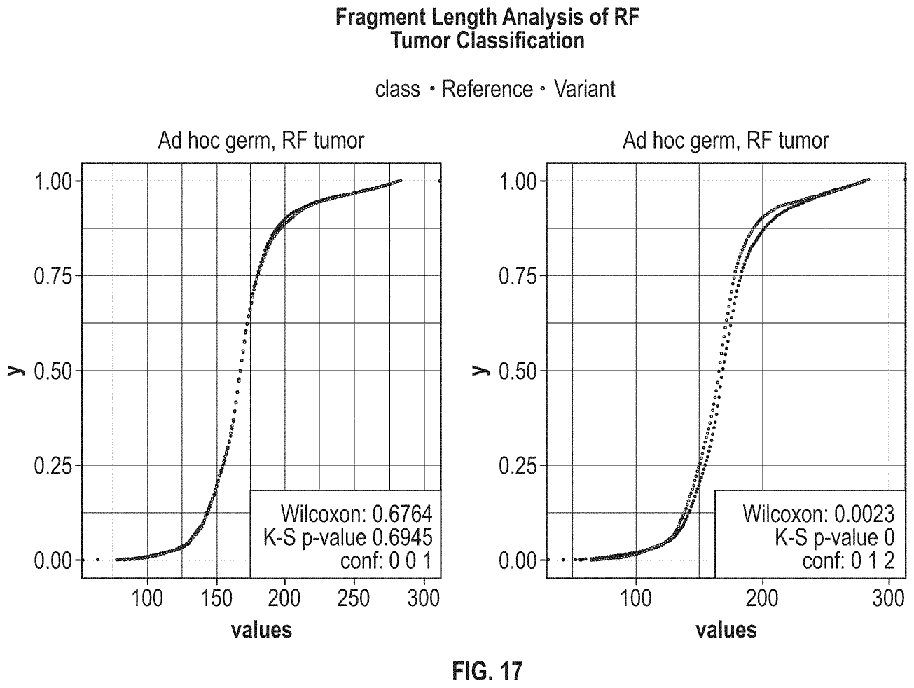

FIG. 17 shows fragment length analysis of variants assigned as originating from tumor by the example classifier, but assigned as germline and other sources by the ad-hoc model. On the one hand, the lack of fragment length shift in the variants sourced as germline by the ad-hoc model and classified as tumor by the classifier (cell count 8 in FIG. 14) indicates that the example classifier is possibly misclassifying these variants. On the other hand, the fragment length shift in the variants sourced as other by the ad-hoc model and classified as tumor by the classifier (cell count 147 in FIG. 14) indicates that the example classifier is likely correctly classifying these variants as tumor-originating.

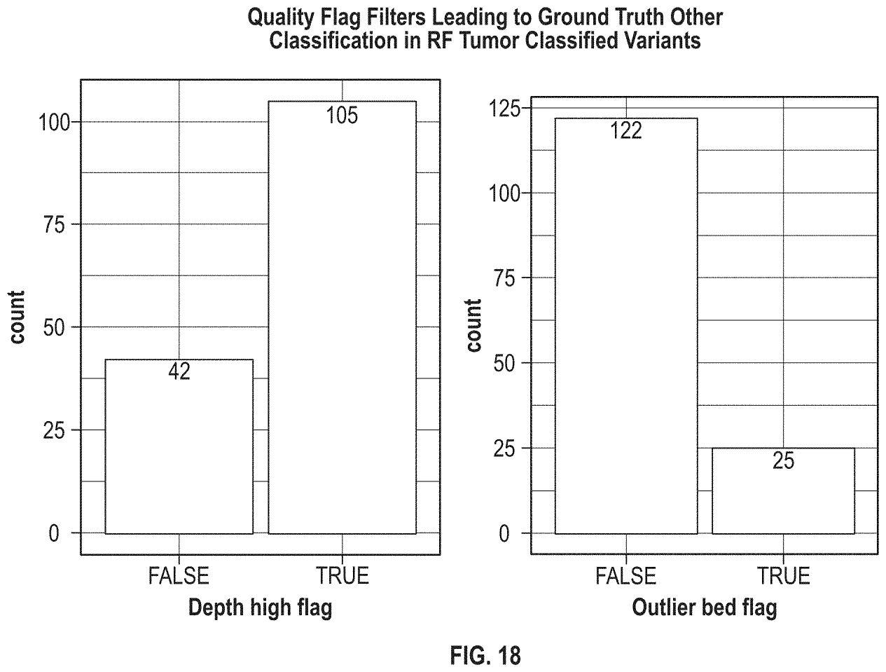

FIG. 18 illustrates plots of decisions made by two ad-hoc filter flags in the ad-hoc model which led to the classification of other by the ad-hoc model for those variants classified as originating from tumor by the example classifier (cell count 147 in FIG. 14).

The left hand plot indicates that most of these variants triggered the “depth high” (true) flag of the ad-hoc model. In the ad-hoc model, cutting off very high depth (above 90^{th} quintile) variants helps avoid classifying variants as coming from a particular source when some of that depth may be arising from segmental duplication or other events that means that the data for that variant is unreliable. As a result, the ad-hoc filter classifies the variant as other. This flag shows one limitation of the ad-hoc model, specifically that some variants will simply have a high depth of data where no interfering effect is causing that high depth. The example classifier is better able to sort through and distinguish these variants as compared to the ad-hoc filter.

The right hand plot indicates that most of these variants triggered the “outlier bed” (false) flag of the ad-hoc model. In the ad-hoc model, some genomic regions of the bed data file, where the bed data file is how training/test data is stored, are flagged as subject to possible mis-mapping. This particularly can happen when the fragments of data processed by the sample processing of FIG. 1 result in shorter than expected fragments. While shorter fragments are generally expected in cancerous cfDNA, generally the amount of shortening is relatively small (e.g., on the order of 20 base pairs), not significantly more. However, if errors occur in sample processing and much shorter fragments are (artificially) identified, due to their shorter length some of these shorter fragments have the possibility of mis-mapping onto the wrong genomic regions. Genomic regions of the bed file where this occurs frequently are referred to as outlier beds. Generally, the ad-hoc model will classify as other variants that fall into these outlier beds. This flag shows another limitation of the ad-hoc model, specifically that some variants will originate in outlier beds, and throwing them out categorically by the ad-hoc model results in some loss of sensitivity.

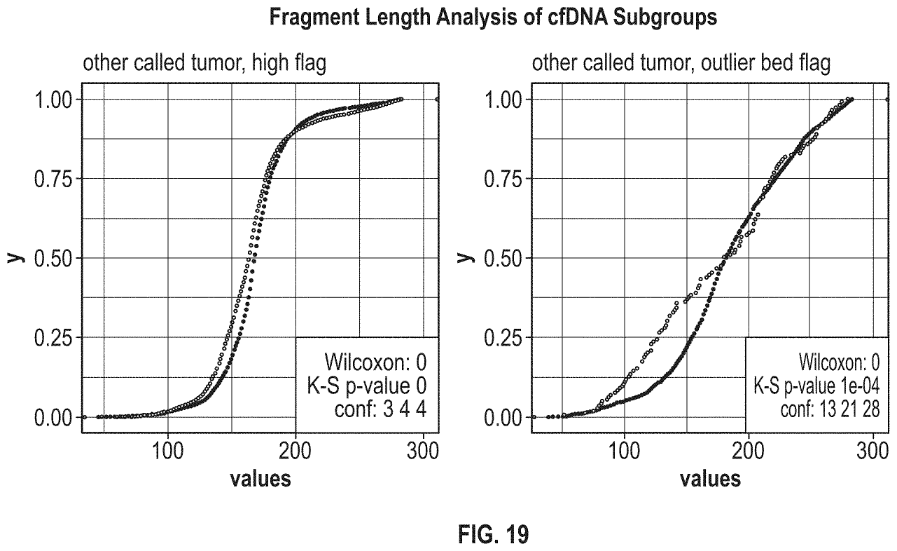

FIG. 19 shows fragment length analyses of variants classified as other by the ad-hoc model and which were classified as tumor by the example classifier (cell count 147 in FIG. 14). The left hand plot shows a fragment length shift for variants called as other by the ad-hoc model as a result of the high depth flag. Here, this fragment length shift indicates that the example classifier is likely correctly classifying many of these variants as from tumor. The left hand plot shows an inconsistent fragment length shift for variants called as other by the ad-hoc model as a result of the outlier bed flag. Here, the shape of the curve for cfDNA data indicates that something in processing is causing the pulling down of reads associated with this variant.

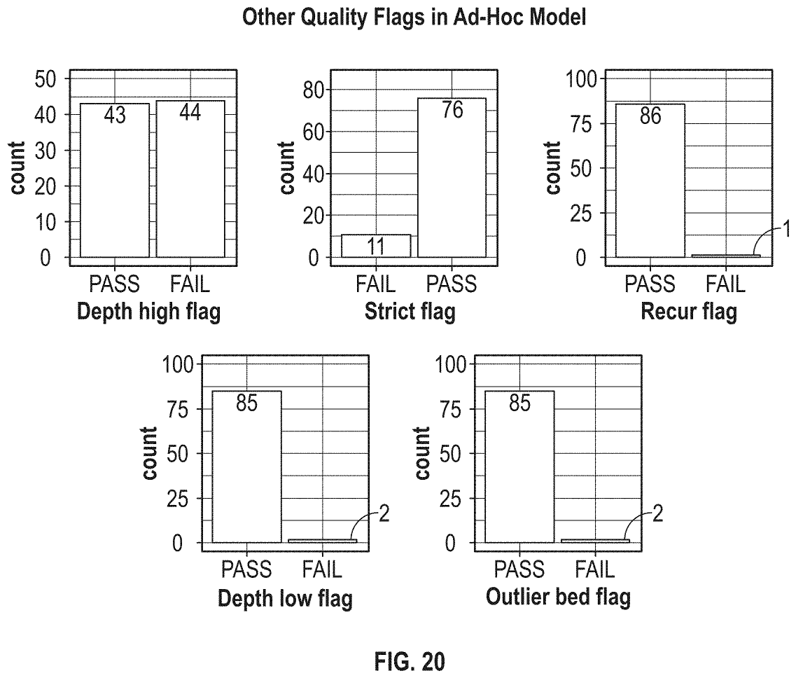

FIG. 20 illustrates plots of decisions made by five other ad-hoc filter flags in the ad-hoc model which led to the classification of other by the ad-hoc model for those variants classified as originating from tumor by the example classifier (cell count 147 in FIG. 14). Here, the depth flag has approximately an equal number of variants pass and fail for those variants the classifier classified as originating from tumor. This suggests that removal of the depth flag from the ad-hoc model will improve sensitivity.

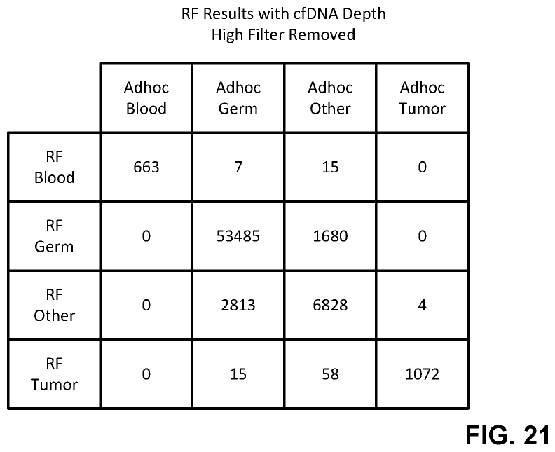

FIG. 21 shows the results of an example alternative ad-hoc model with the depth high flag and with the same example source assignment classifier as discussed in this example. The layout of the results table is the same as in FIG. 14. In comparison to the results of FIG. 14, the table of FIG. 21 shows significantly fewer variants classified by the ad-hoc model as other. Instead, the counts of the number of variants classified by the ad-hoc model as from blood, germline, and tumor have all increased. This suggests that the removal of the high depth flag has improved the ad-hoc model's sensitivity and specificity. However, there are still a number of variants (58) classified as other by the ad-hoc model which the example classifier classifies as from tumor. This suggests that the example classifier still outperforms the ad-hoc model despite the improvement.

### VI.E. Example 3: Evaluation of gDNA Covariates in the Source Assignment Classifier

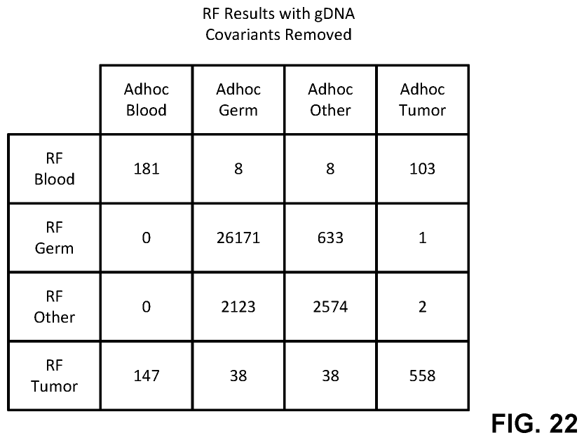

In this example, the example RFC source assignment classifier discussed in example 2 was trained with all of the covariates from Table 1 except it omitted the entire gDNA set of covariates from Table 1. FIG. 22 shows the results of the removal of gDNA covariates for this example classifier. These results show a drop in sensitivity and specificity relative to the performance of the example classifier from example 2, illustrated in FIG. 14 above. Specifically, the example classifier classifies a significant number of variants as blood vs. the ad-hoc model calling them as tumor.

### VII. Additional Considerations

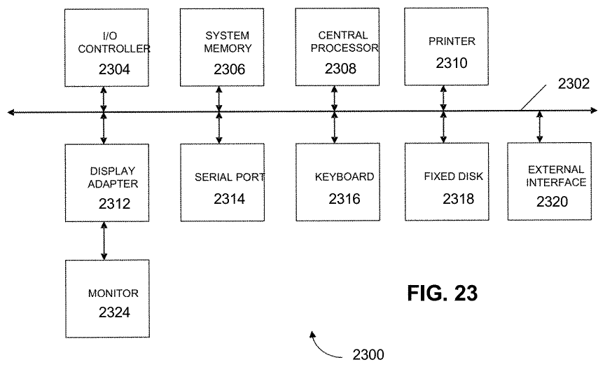

Turning now to FIG. 23, components of an example computer system 2300 utilized in performing the systems and methods described herein are shown. Any of the systems mentioned herein for performing any of the methods can utilize any suitable number of subsystems. In some embodiments, a computer system includes a single computer apparatus, where the subsystems can be the components of the computer apparatus. In some embodiments, a computer system can include multiple computer apparatuses, each being a subsystem, with internal components.

The subsystems shown in FIG. 23 are interconnected via a system bus 2302. Additional subsystems such as a printer 2310, keyboard 2316, fixed disk 2318, monitor 2324, which is coupled to display adapter 2312, and others are shown. Peripherals and input/output (I/O) devices, which couple to I/O controller 2304, can be connected to the computer system by any number of means known in the art, such as serial port 2314. For example, serial port 2314 or external interface 2320 (e.g. Ethernet, Wi-Fi, etc.) can be used to connect computer system 2300 to a wide area network such as the Internet, a mouse input device, or a scanner. The interconnection via system bus 2302 allows the central processor 2308 to communicate with each subsystem and to control the execution of instructions from system memory 2306 or the fixed disk 2318, as well as the exchange of information between subsystems. The system memory 2306 and/or the fixed disk 2318 can embody a computer readable medium. Any of the values mentioned herein can be output from one component to another component and can be output to the user.

A computer system can include a plurality of the same components or subsystems, e.g., connected together by external interface 2320 or by an internal interface. In some embodiments, computer systems, subsystem, or apparatuses can communicate over a network. In such instances, one computer can be considered a client and another computer a server, where each can be part of a same computer system. A client and a server can each include multiple systems, subsystems, or components.

The computing device/s introduced above (e.g., FIG. 23) can be one of a server computer, personal computer (PC), a desktop computer, a laptop computer, a notebook, a tablet PC, a mobile device. A computing device can be communicatively coupled to one or more sequencers providing the output file through a wireless, wired, or a combination of wireless and wired communication technologies. Generally, the computing devices are configured with a processor and memory storing computer instructions that, when executed by the processor, cause the processor to carry out the computation analyses described below including the source assignment classification methods described herein.

The computing devices generally, though not always, include one or more of the following: processing units CPU(s) (also referred to as processors), graphical processing units, network interfaces, a graphical user interface displayed on a display screen associated with the computing device, a non-persistent memory, a persistent memory, and a communication bus for interconnecting these components. The communication bus optionally include circuitry (sometimes called a chipset) that interconnects and controls communications between system components.

The non-persistent memory typically includes high-speed random access memory, such as DRAM, SRAM, DDR RAM, ROM, EEPROM, flash memory, whereas the persistent memory typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices.

The persistent memory optionally includes one or more storage devices remotely located from the CPU(s). The persistent memory, and the non-volatile memory device(s) within the non-persistent memory, comprise non-transitory computer readable storage medium.

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like.

Any of the steps, operations, or processes described herein may be performed or implemented with one or more hardware or software modules, alone or in combination with other devices. In one embodiment, a software module is implemented with a computer program product including a computer-readable non-transitory medium containing computer program code, which can be executed by a computer processor for performing any or all of the steps, operations, or processes described.

Embodiments of the invention may also relate to a product that is produced by a computing process described herein. Such a product may include information resulting from a computing process, where the information is stored on a non-transitory, tangible computer-readable storage medium and may include any embodiment of a computer program product or other data combination described herein.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention.

1. A method for determining a source of a variant comprising:
receiving a plurality of variants obtained from a biological sample, the variants being of unknown source upon receipt;
receiving, for each of the variants, a plurality of values for a plurality of covariates from the biological sample;
inputting the variants into a source assignment classifier to determine a source for each of the variants, the source being one of a plurality of possible sources, the source assignment classifier comprising:
a plurality of coefficients associated with the plurality of covariates and
a function receiving as input the values associated with each variant and the coefficients and outputting the determined source of each of the variants;
providing the determined sources for the variants.

2. The method of claim 1, wherein determining the source for each of the variants comprises determining a numerical score for the determined source associated with each variant.

3. The method of claim 2, wherein determining the source for each of the variants further comprises determining a numerical confidence value associated with the numerical score.

4. The method of claim 2, wherein determining the source for each of the variants further comprises determining a numerical score for each of the possible sources.

5. The method of claim 4, wherein determining the source for each of the variants further comprises determining a numerical confidence value associated with each of the numerical scores associated with each of the possible sources.

6. The method of claim 1, wherein the plurality of possible sources include at least one of: a tumor source, a germline source, a blood source, an other source, and an unknown source.

7. The method of claim 6, wherein the source assignment classifier assigns variants to the unknown source where confidences in other sources are below at least one threshold.

8. The method of claim 1, wherein the values include information regarding cfDNA sequencing data, and the covariates includes at least one covariate indicating an accuracy of cfDNA sequencing for the variants.

9. The method of claim 8, wherein one or more of the covariates comprises one or more of:
a count of variant reads in cfDNA,
a count of reference reads in cfDNA,
a total count of reference reads and variant reads in cfDNA,
a variant allele frequency in cfDNA of one of the variants input into the model,
a cfDNA sequencing quality score derived from a noise model, and
a strand bias in the cfDNA sequencing quality score.

10. The method of claim 1, wherein the values include information regarding gDNA sequencing data, and the covariates includes at least one covariate indicating an accuracy of gDNA sequencing for the variants.

11. The method of claim 10, wherein one or more of the covariates comprises one or more of:
a count of variant reads in gDNA, a count of reference reads in gDNA,
a total count of reference reads and variant reads in gDNA,
a variant allele frequency in gDNA,
a gDNA sequencing quality score derived from a noise model,
an indication of a presence or absence of the variant in gDNA greater than a threshold,
a count of reads of a variant with overlapping positions in pileup data in gDNA,
a count of variant reads in pileup data in gDNA, and
a count of reference reads in pileup data in gDNA.

12. The method of claim 1, wherein one of the covariates indicates if one of the variants recurs above a threshold frequency.

13. The method of claim 1, wherein one of the covariates is a ratio of a variant allele frequency in gDNA to a variant allele frequency in cfDNA, or vice versa.

14. The method of claim 1, wherein one of the covariates indicates a category of an allele change from one allele to another allele.

15. The method of claim 1, wherein one of the covariates indicates a trinucleotide context of one of the variants.

16. The method of claim 1, wherein one of the covariates indicates if a position of one of the variants overlaps a segmental duplication.

17. The method of claim 1, wherein one of the covariates indicates if a gene associated with one of the variants overlaps a known clonal hematopoiesis gene.

18. The method of claim 1, wherein one of the covariates indicates if a threshold number of mapping locations overlap a position of one of the variant.

19. A non-transitory computer-readable storage medium storing one or more programs configured to be executed by one or more processors of an electronic device, the one or more programs including instructions for:
receiving a plurality of variants obtained from a biological sample, the variants being of unknown source upon receipt;
receiving, for each of the variants, a plurality of values for a plurality of covariates from the biological sample;
inputting the variants into a source assignment classifier to determine a source for each of the variants, the source being one of a plurality of possible sources, the source assignment classifier comprising:
a plurality of coefficients associated with the plurality of covariates and
a function receiving as input the values associated with each variant and the coefficients and outputting the determined source of each of the variants;
providing the determined sources for the variants.

20. An electronic device comprising:
one or more processors;
a memory; and
one or more programs, wherein the one or more programs are stored in the memory and configured to be executed by the one or more processors, the one or more programs including instructions for:
receiving a plurality of variants obtained from a biological sample, the variants being of unknown source upon receipt;
receiving, for each of the variants, a plurality of values for a plurality of covariates from the biological sample;
inputting the variants into a source assignment classifier to determine a source for each of the variants, the source being one of a plurality of possible sources, the source assignment classifier comprising:
a plurality of coefficients associated with the plurality of covariates and
a function receiving as input the values associated with each variant and the coefficients and outputting the determined source of each of the variants;
providing the determined sources for the variants.
